(19) United States
(12) Patent Application Publication
(10) Pub. No.: US 2023/0232851 A1
(43) Pub. Date: Jul. 27, 2023

(54) METHOD OF REDUCING GROWTH OF LISTERIA IN FOOD PRODUCTS

(71) Applicant: Chr. Hansen A/S, Hoersholm (DK)

(72) Inventors: Mikkel Gulmann Madsen, Hoersholm (DK); Helle Skov Guldager, Hoersholm (DK); Louise Essendrup Steffensen, Hoersholm (DK); Shannon Neuens, Milwaukee, WI (US); Anja Kristiansen, Hoersholm (DK); Cecilie Lykke Marvig Nielsen, Hoersholm (DK); Dana Wolle, Milwaukee, WI (US)

(73) Assignee: Chr. Hansen A/S, Hoersholm (DK)

(21) Appl. No.: 17/919,004

(22) PCT Filed: Apr. 16, 2021
(86) PCT No.: PCT/EP2021/059910

§ 371 Date: Oct. 14, 2022

(30) Foreign Application Priority Data
Apr. 16, 2020 (EP) 20169838.8

(51) Int. Cl. A23C 19/06; A23C 19/076; A23C 19/068; C12N 1/20

(52) U.S. Cl. CPC A23C 19/062; A23C 19/076; A23C 19/0684; C12N 1/205; A23Y 2220/73; C12R 2001/225

(57) ABSTRACT

The present invention is in the field of dairy technology. It relates to methods of inhibiting Listeria in dairy products, characterized in that the fermentation is carried out using Lactobacillus rhamnosus DSM 32092 or a mutant thereof. The invention also provides use of Lactobacillus rhamnosus DSM 32092 to inhibit Listeria and dairy products comprising Lactobacillus rhamnosus DSM 32092 such as cheeses.

### FIELD OF THE INVENTION

The present invention relates to methods of inhibiting the growth of Listeria during the production of fermented milk products, in particular cheese products. The methods of the present invention comprise a step of fermenting milk with a culture comprising Lactobacillus rhamnosus. In particular, the present invention relates to methods for the production of dairy products, in particular cheese, using a culture of Lactobacillus rhamnosus.

### BACKGROUND OF THE INVENTION

Bacterial contamination of food products is known to be responsible for spoilage and for the transmission of food borne illnesses. The problem may arise in particular with dairy food products which are not normally reheated by consumers prior to ingestion and which are stored for an extended period of time in a refrigerator at 2-10° C. For example, Listeria monocytogenes is a pathogenic bacterium of particular concern in food products. It can grow in environments with a pH between 4.3 and 9.5. Tolerance of L. monocytogenes to pH is also linked with the water activity (aw). It is commonly reported that the bacterium can grow at an aw higher than 0.92. Therefore, with respect to pH level, there are many foods that seem susceptible to the growth of L. monocytogenes. It is known that Listeria tolerates refrigeration temperatures, relatively high concentrations of NaCl and anaerobic conditions in dairy food products, such as cheese. Listeriosis is a bacterial infection caused by Listeria monocytogenes and is a known cause for severe illness, including severe sepsis, meningitis, or encephalitis, sometimes resulting in lifelong harm and even death. At risk of severe illness are in particular the elderly, unborn babies, newborns and immunocompromised persons. In pregnant women listeriosis may cause stillbirth or spontaneous abortion, and preterm birth is common. Listeriosis may cause mild, self-limiting gastroenteritis and fever. As a consequence, a significant effort has been made to inhibit the growth of Listeria monocytogenes in food products.

Lactic acid bacteria have been used for inhibiting pathogens and/or increasing the shelf life of food products. During fermentation lactic acid bacteria produce organic acids and thus reduce the pH of the fermented food product. This inhibits the growth of unwanted microorganisms, such as pathogenic bacteria as well as yeasts and fungi.

European Patent Application 1 442 113 discloses mixtures of Propionibacterium jensenii and Lactobacillus sp., such as Lactobacillus paracasei, with antimicrobial activity for use for bioprotection.

WO 2017/050980 relates to the use of Lactobacillus rhamnosus and/or supernatants thereof for the inhibition of pathogens, such as Salmonella enteritidis, Pseudomonas aeruginosa, Escherichia coli, Listeria monocytogenes, Clostridium difficile and Enterococcus faecalis. However, no data for Listeria was shown.

WO 2017/037046 discloses Lactobacillus fermentum strains with antifungal activity. A synergistic antifungal effect was further observed when carrying out fermentations using a combination of Lactobacillus fermentum and Lactobacillus rhamnosus in comparison to the use of each.

Tharmaraj et al. co-cultured several probiotic L rhamnosus (LC705, LBA, LGG and LR1524) in cheese based-dip with some pathogenic bacteria, including E. coli, S. typhimurim, S. aureus and B. cereus. The author observed a reduction of the bacterial population was observed in the control probably due to the acidity of the dip (pH 4.3). In the tested dips, the probiotic bacteria played a limited role in inhibiting pathogenic bacteria E. coli, S. typhimurium and S. aureus. No test were done against Listeria (Tharmaraj et al. “Antimicrobial effects of probiotics against selected pathogenic and spoilage bacteria in cheese-based dips.” International Food Research Journal 16.1 (2009): 261-276). The European Commission (EC) has established criteria to define the acceptability of a ready-to-eat (RTE) food, based on the presence/absence or enumeration of L. monocytogenes throughout the food supply chain for a given type of food.

There is a need for bioprotective agents with anti-Listerial effects which can be used for inhibiting growth of Listeria in dairy products at a pH higher than 4.3 or an aw above 0.92 to comply with regulations.

The use of raw milk is one of the major factors for the contamination with L. monocytogenes in dairy products. Milk heat treatment is sometimes insufficient to guarantee the absence of L. monocytogenes in dairy products. It is known that a lack of hygiene or sanitation during the post-pasteurization or post-processing steps would also lead to contamination.

There is in particular a need for bioprotective agents with anti-Listerial effects which can be used for inhibiting growth of Listeria during the production of dairy food products, such as maturation in the cases for cheeses, as they are subject to Listeria contamination.

### SUMMARY OF THE INVENTION

Dairy products with a high pH and high water activity are prone to Listerial contaminations, which may lead to foodborne diseases if not controlled. It has been surprisingly discovered that Lactobacillus rhamnosus DSM 32092 or a mutant thereof can be used to reduce the growth of Listeria in dairy products with high pH and high water activity. This is the first time that anti-Listerial activity of DSM 32092 has been identified.

In a first aspect, the present invention provides a method for inhibiting Listeria growth in dairy products using Lactobacillus rhamnosus DSM 32092 or a mutant thereof. In one aspect, the present invention provides a method for inhibiting the growth of Listeria during the production and/or shelf life of dairy products, in particular cheese products.

As used herein, “during the production” refers to the period when the products are being manufactured and made ready for distribution to customers. In one embodiment, the product is a cheese that needs ripening, and this period includes maturation time for ripening. The methods comprise a step of fermenting milk or milk substitute with a culture comprising Lactobacillus rhamnosus DSM 32092 or a mutant of Lactobacillus rhamnosus DSM 32092. In some embodiments, the mutant has at least 75% of the anti-Listerial activity of Lactobacillus rhamnosus DSM 32092, such as at least 80%, 85%, 90%, 92%, 95%, 98% or 99% of the anti-Listerial activity of Lactobacillus rhamnosus DSM 32092.

In one embodiment, the Listeria bacteria inhibited by the methods of the present invention are bacteria of the species Listeria monocytogenes and Listeria innocua.

The methods of the present invention can be used to inhibit the growth of Listeria for any type of cheeses. However, cheeses that are susceptible to Listeria contamination are of particular focus. The methods disclosed herein can be used in the production of cheeses having higher pH (>4.3) and high water activity (0.92). This includes fresh cheese, soft cheese, semisoft cheese, and a few types of hard and semihard cheeses. As L. monocytogenes is halotolerant, able to grow in concentration of salt up to 10.0%, low salt cheese is also particularly susceptible to contamination.

In preferred embodiments, the methods of the present invention are suitable for inhibiting the growth of Listeria during the production and the shelf life of soft and semisoft cheese. Cottage cheese or Mozzarella cheese is particularly preferred.

The methods of the present invention comprise a step of fermenting milk or milk substrate with a culture comprising L. rhamnosus DSM 32092 or a mutant thereof. In a preferred embodiment, the milk or milk substrate is inoculated with the L. rhamnosus DSM 32092 in a concentration of at least 1×10⁶ CFU/mL, such as at least 1×10⁷ CFU/mL, such as at least 1×10⁸ CFU/mL, for example in a concentration range of 1×10⁶ to 1×10⁸ CFU/mL, preferably in a concentration range of 1×10⁷ to 1×10⁸ CFU/mL or preferably in a concentration of 5×10⁷ to 1×10⁸ CFU/mL. In a further embodiment, the milk or milk substrate is inoculated with L. rhamnosus DSM 32092 and a primary starter culture which is a dairy starter culture.

For example, the primary starter culture can be any culture commonly used for cheese production. A skilled person is able to select suitable starter culture depending on the cheese to be produced. In a preferred embodiment, the starter culture comprises lactic acid bacteria. In some embodiments, the starter culture comprises Lactococcus spp., Streptococcus spp., Leuconostoc spp., and/or Lactobacillus spp. In some embodiments, the starter culture comprises Lactococcus lactis subsp. cremoris, Lactococcus lactis subsp. lactis, Lactococcus lactis subsp. lactis biovar. diacetylactis, Streptococcus thermophilus, Leuconostoc spp., Lactobacillus delbrueckii subsp. bulgaricus, and Lactobacillus helveticus

In some embodiments, the starter culture comprises Lactococcus lactis subsp. lactis and Streptococcus thermophilus, including a starter culture consisting of Lactococcus lactis subsp. lactis and Streptococcus thermophilus, such as the culture commercially available from Chr. Hansen A/S Denmark as FRESCO 1000NG-10. The starter culture may comprise Lactococcus lactis subsp. lactis, Streptococcus thermophilus, and Lactococcus lactis subsp. cremoris.

In some embodiments, the starter culture comprises Streptococcus thermophilus.

In some embodiments, the starter culture comprises Lactobacillus delbrueckii subsp. bulgaricus.

In some embodiments, the starter culture comprises Lactococcus spp., such as Lactococcus lactis subsp. lactis and Lactococcus lactis subsp. cremoris.

In some embodiments, the starter culture comprises Lactococcus spp. and Leuconostoc spp., such as Lactococcus lactis subsp. lactis and Lactococcus lactis subsp. cremoris, Lactococcus lactis subsp. lactis biovar. diacetylactis and Leuconostoc.

Starter cultures are commercially available, such as FRESCO, WhiteDaily, WhiteFlora, WhiteClassic, SOFT CREMOSO, CZ, SOFT FLORA, MO, CHN, FloraDanica, LB Stella, i400, STi from Chr. Hansen A/S Denmark.

In a further embodiment the starter culture used for the production of cheese does not comprise bacteria of the species Lactobacillus fermentum.

The present invention is also related to the use of bacteria of the strain Lactobacillus rhamnosus DSM 32092 or a mutant of Lactobacillus rhamnosus DSM 32092, wherein the mutant maintains at least 75% of the anti-Listerial activity of Lactobacillus rhamnosus DSM 32092 to inhibit the growth of Listeria. Inhibition may be determined according to the assay as described herein.

The present invention further provides a cheese, such as cottage cheese or pasta filata cheese, comprising bacteria of the strain Lactobacillus rhamnosus DSM 32092 or a mutant of Lactobacillus rhamnosus DSM 32092, wherein the mutant maintains at least 75% of the anti-Listerial activity of Lactobacillus rhamnosus DSM 32092.

### Definitions

As used herein, the term “lactic acid bacteria” designates a gram-positive, microaerophilic or anaerobic bacterium, which ferments sugars with the production of acids including lactic acid as the predominantly produced acid.

The term “dairy product” refers to any raw and/or processed dairy material or material derived from dairy ingredients. It is meant to include also cheese. “Cheese” refers to a product prepared by contacting milk, optionally acidified milk, such as milk that is acidified e.g. by means of a lactic acid bacterial culture and optionally with a coagulant and draining the resultant curd. The term “cheese” includes any form of cheese, such as natural cheese, cheese analogs, cheese (processed cheese). A person skilled in the art knows how to convert the coagulum, also known as curd, into cheese, methods can be found in the literature, see e.g. Kosikowski, F. V., and V. V. Mistry, “Cheese and Fermented Milk Foods”, 1997, 3^{rd} Ed. F. V. Kosikowski, L. L. C. Westport, Conn.

Lactic acid bacteria, including bacteria of the species Lactobacillus, Streptococcus, Leuconostoc and Lactococcus, are normally supplied to the dairy industry either as frozen or freeze-dried cultures for bulk starter propagation or as so-called “Direct Vat Set” (DVS) cultures, intended for direct inoculation into a fermentation vessel or vat for the production of a dairy product, such as a fermented milk product or a cheese. Such lactic acid bacterial cultures are in general referred to as “starter cultures” or “starters”. Starter cultures may be liquid, frozen or freeze-dried. In one embodiment the starter culture is bulk starter culture. It is within the skills of ordinary practitioners to determine the starter culture and amounts to be used.

The starter culture composition may comprise the bacteria in a concentrated form including liquid, frozen, dried or freeze-dried concentrates typically having a concentration of viable cells, which is in the range of 10⁴ to 10¹² cfu (colony forming units) per gram of the composition including at least 10⁴ cfu per gram of the composition, such as at least 10⁵ cfu/g, e.g. at least 10⁶ cfu/g, such as at least 10⁷ cfu/g, e.g. at least 10⁸ cfu/g, such as at least 10⁹ cfu/g, e.g. at least 10¹⁰ cfu/g, such as at least 10¹¹ cfu/g.

The term “milk” is to be understood as the lacteal secretion obtained by milking of any mammal, such as cows, sheep, goats, buffaloes or camels. Milk base can be obtained from any raw and/or processed milk material as well as from reconstituted milk powder. Milk base can also be plant-based, i.e. prepared from plant material e.g. soy milk, almond milk, cashew milk or coconut milk. Milk base prepared from milk or milk components from cows is preferred. In some preferred embodiments, the milk is raw milk (i.e. unpasteurized) obtained from cows, sheep, goats, buffaloes or camels.

The term “milk substrate” may be any raw and/or processed milk material that can be subjected to fermentation according to the method of the invention. Thus, useful milk substrates include, but are not limited to, solutions/suspensions of any milk or milk like products comprising protein, such as whole or low fat milk, skim milk, buttermilk, reconstituted milk powder, condensed milk, dried milk, whey, whey permeate, lactose, mother liquid from crystallization of lactose, whey protein concentrate, or cream. Obviously, the milk substrate may originate from any mammal, e.g. being substantially pure mammalian milk, or reconstituted milk powder.

Prior to fermentation, the milk substrate may be homogenized and pasteurized according to methods known in the art.

“Homogenizing” as used herein means intensive mixing to obtain a soluble suspension or emulsion. If homogenization is performed prior to fermentation, it may be performed so as to break up the milk fat into smaller sizes so that it no longer separates from the milk. This may be accomplished by forcing the milk at high pressure through small orifices.

“Pasteurizing” as used herein means treatment of the milk substrate to reduce or eliminate the presence of live organisms, such as microorganisms. Preferably, pasteurization is attained by maintaining a specified temperature for a specified period of time. The specified temperature is usually attained by heating. The temperature and duration may be selected in order to kill or inactivate certain bacteria, such as harmful bacteria. A rapid cooling step may follow. Raw milk refers to milk which is not pasteurized.

“Fermentation” in the methods of the present invention means the conversion of carbohydrates into alcohols or acids through the action of a microorganism. Preferably, fermentation in the methods of the invention comprises conversion of lactose to lactic acid.

Fermentation processes to be used in production of dairy products are well known and the person of skill in the art will know how to select suitable process conditions, such as temperature, oxygen, amount and characteristics of microorganism(s) and process time. Fermentation conditions are selected so as to support the achievement of the present invention, i.e. to obtain a dairy product in solid (such as a cheese) or liquid form (such as a fermented milk product). Depending on the product to be produced, fermentation can be for example between 4 to 12 hours, such as 6-10 hours. Fermentation can be stopped when a desired pH is reached.

“Inhibiting” generally means a decrease, whether partial or whole, in function and activity of cells or microorganisms. As used herein, the terms “to inhibit” or “inhibiting” in relation to bacteria, yeasts or molds mean that the growth is reduced. This can be measured by any methods known in the field of microbiology. Inhibition can be observed by comparing the growth, number or concentration to a reference value or a control. The control can be the same product prepared the same way but without using DSM 32092. The terms “to inhibit” and “to be inhibiting” in relation to Listeria mean for example that the growth or the number or Listeria is lower than in the product which is prepared the same way but without Lactobacillus rhamnosus DSM 32092.

In the present context, the term “mutant” should be understood as a strain derived from a strain of the invention by means of e.g. genetic engineering, radiation and/or chemical treatment. It is preferred that the mutant is a functionally equivalent mutant, e.g. a mutant that has substantially the same, or improved, properties (e.g. regarding anti-Listeria properties) as the mother strain. Such a mutant is a part of the present invention. Especially, the term “mutant” refers to a strain obtained by subjecting a strain of the invention to any conventionally used mutagenization treatment including treatment with a chemical mutagen such as ethane methane sulphonate (EMS) or N-methyl-N′-nitro-N-nitroguanidine (NTG), UV light or to a spontaneously occurring mutant. A mutant may have been subjected to several mutagenization treatments (a single treatment should be understood one mutagenization step followed by a screening/selection step), but it is presently preferred that no more than 20, or no more than 10, or no more than 5, treatments (or screening/selection steps) are carried out. In a presently preferred mutant, less than 5%, or less than 1% or even less than 0.1% of the nucleotides in the bacterial genome have been shifted with another nucleotide, or deleted, compared to the mother strain.

One advantage of the present invention is the to ensure food safety by controlling growth of Listeria during the shelf life of dairy products. As used herein, the term “shelf life” means the period of time that a food product remains sellable to retail customers.

### BRIEF DESCRIPTION OF THE FIGURES

FIG. 1

FIG. 1 shows the level of Listeria monocytogenes in cottage cheese fermented with the starter culture Fresco and L. rhamnosus DSM 32092, Fresco and LMG18030, Fresco 1000NG-10 and LMG18025, Fresco 1000NG-10 and LMG18020, Fresco 1000NG-10 and CNCM I-4316, or Fresco 1000NG-10 alone (control). Listeria monocytogenes was added to the cottage cheese in the concentration of around 1×10⁴ CFU/g. The growth of Listeria monocytogenes was determined for a period of 2, 7, 12, 16 or 20 days, respectively.

FIG. 2

FIG. 2 presents the same data as seen FIG. 1. Growth curve of Listeria monocytogenes is shown at the period of 2, 7, 12, 16 or 20 days for each sample.

FIG. 3

FIG. 3 shows the cooling profile for cottage cheese prepared in Example 2.

FIG. 4

FIG. 4 shows the growth change of Listeria monocytogenes in the cottage cheese prepared with and without DSM 32092.

FIG. 5

FIG. 5 shows the growth change of Listeria monocytogenes in Mozzarella cheese prepared with and without DSM 32092.

### DETAILED DESCRIPTION OF THE INVENTION

The present invention provides new methods to inhibit the growth of Listeria in dairy products. In particular, the present invention provides methods for inhibiting the growth of Listeria in dairy products, preferably cheese products, characterized in that Lactobacillus rhamnosus DSM 32092 or a mutant thereof is used. The methods comprise a step of fermenting milk or milk substrate with a culture comprising Lactobacillus rhamnosus DSM 32092 or a mutant of Lactobacillus rhamnosus DSM 32092, wherein the mutant maintains at least 75% of the anti-Listerial activity of Lactobacillus rhamnosus DSM 32092.

Cases of human listeriosis are almost exclusively caused by the species L. monocytogenes. Listeria monocytogenes can be divided into 13 different serotypes all of which are able to cause listeriosis. However, most cases are caused by serotypes 1/2a, 1/2b and 4b.

Bacteria of the genus Listeria can be of the species Listeria monocytogenes or Listeria innocua. L. innocua and L. Listeria have been found to behave similarly in dairy environment. Listeria innocua is generally considered nonpathogenic and is used as surrogate in pilot studies which reflect and predict inhibition of Listeria monocytogenes. In addition, a fatal case of Listeria innocua bacteremia has been reported (Perrin et al, Journal of Clinical Microbiology 41.11 (2003): 5308-5309). It is therefore envisioned that bacteria of the genus Listeria can be of the species Listeria innocua. The methods of the present invention can be used for manufacturing many types of dairy products, such as yoghurt or cheese. Cheeses are of particular focus because they are susceptible to Listeria contamination. The methods disclosed herein can be used manufacture cheese products with high pH (>4.3) and/or high water activity (>0.92). This includes fresh cheese, soft cheese, semisoft cheese, and a few types of hard and semihard cheeses.

In preferred embodiments, the dairy product has a pH which is higher than 4.3 but lower than 7.0, such as higher than 4.4, such as higher than 4.5, such as higher than 4.6, such as higher than 4.7, such as higher than 4.8, such as higher than 4.9, such as higher than 5.0, such as higher than 5.1, such as higher than 5.2, such as higher than 5.3, such as higher than 5.4, such as higher than 5.5, such as higher than 5.6, such as higher than 5.7, such as higher than 5.8, such as higher than 5.9, such as higher than 6.0, such as higher than 6.1, such as higher than 6.2, such as higher than 6.3, such as higher than 6.4, such as higher than 6.5, such as higher than 6.6, such as higher than 6.7, such as higher than 6.8, such as higher than 6.9.

In preferred embodiments, the dairy product has a water activity higher than 0.92, such as higher than 0.93, such as higher than 0.94, such as higher than 0.95, such as higher than 0.96, such as higher than 0.97, such as higher than 0.98, such as higher than 0.99.

With its low pH, yoghurt is less prone to Listerial contamination. However, a potential health hazard could arise if a sufficiently high amount of L. monocytogenes recontaminates milk after heat treatment in small plants where unsophisticated methods are used.

Dairy products or cheese products prepared by the methods described in the present application may be characterized in that the level of Listeria is lower than the product prepared with the same method but without using Lactobacillus rhamnosus DSM 32092. LISTERIA DETECTION The level of Listeria can be determined using routine enumeration methods known in the art. One may apply standard protocols in US FDA's Bacteriological Analytical Manual (BAM) (Hitchins et al., “BAM: Detection and Enumeration of Listeria monocytogenes.” Bacteriological analytical manual (2016)) or protocols published by the European and International Standard method EN ISO 11290-1:2017 (ISO, PNEN. “11290-1: 2017. Microbiology of the food chain-Horizontal method for the detection and enumeration of Listeria monocytogenes and of Listeria spp.”). Other methods can also be used, such as described in Law et al. “An insight into the isolation, enumeration, and molecular detection of Listeria monocytogenes in food.” Frontiers in microbiology 6 (2015): 1227.

In some embodiments, dairy products prepared using the methods described in the present application may have a Listeria count of less than 100 cfu/g during the shelf life, for example, at day 1, 5, 10, 15, 20, 25, 30, 35, 40, 45, 50, 55 or 60 days, when stored at a temperature between 1-8° C.

In some embodiments, the cheese products prepared using the methods described in the present application may have a Listeria count of less than 100 cfu/g at day 1, 2, 3, 4, 5, 6, 7, 8, 9, 10, 11, 12, 13, 14, 15, 16, 17, 18, 19, 20, 21, 22, 23, 24, 25, 26, 27, 28, 29 or day 30 when stored at a temperature between 1-8° C.

The methods of producing a dairy product according to the present invention can be a characterized by a temperature during fermentation of between 22 and 45° C. This temperature range includes the range used for mesophilic and thermophilic cultures. In the context of the present application the term “mesophilic” refers to microorganisms that grow best at moderate temperatures, i.e. at temperatures from 15° C. to 40° C. The industrially most useful mesophilic bacteria include Lactococcus spp. and Leuconostoc spp. Mesophilic dairy products include such dairy products as buttermilk, sour milk, cultured milk, smetana, sour cream and fresh cheese, such as quark, tvarog and cream cheese. In the context of the present application the term “thermophilic” refers to microorganisms that grow best at temperatures above 40° C. The industrially most useful thermophilic bacteria include Streptococcus spp. and Lactobacillus spp. Thermophilic dairy products include such dairy products as yoghurt.

In particular, term “yoghurt” encompasses, but is not limited to, yoghurt as defined according to French and European regulations, e.g. coagulated dairy products obtained by lactic acid fermentation by means of specific thermophilic lactic acid bacteria only (i.e. Lactobacillus delbrueckii subsp. bulgaricus and Streptococcus thermophilus) which are cultured simultaneously and are found to be live in the final product in an amount of at least 10 million CFU (colony-forming unit)/g.

The dairy products of the present invention are preferably fermented dairy products, including yoghurt, fruit yoghurt, yoghurt beverage or cheese.

In its most preferred embodiment, all methods of the present invention are methods for producing a cheese and the product of the present invention is cheese.

“Cheese product” is a term defined in accordance with relevant official regulations. The standards for such products are well known in the field. The method as disclosed herein are especially applicable to cheese products with a pH which higher than 4.3 and/or a water activity 0.92. In preferred embodiments, the dairy product of the present application is soft or semisoft cheese. In a further embodiment, the cheese is cottage cheese, such as warm-filled cottage cheese, or Mozzarella cheese as described in the examples. Such cheese is characterized by higher packaging temperatures and longer cooling times. Contamination is more likely to occur during filling from filling equipment at higher temperature.

The present invention is also related to the use of bacteria of the strain Lactobacillus rhamnosus DSM 32092 or a mutant of Lactobacillus rhamnosus DSM 32092, wherein the mutant maintains at least 75%, such as at least 80%, 85%, 90%, 92%, 95%, 98% or 99% of the anti-Listerial activity of Lactobacillus rhamnosus DSM 32092, for the inhibition of Listeria. In one embodiment, Listeria is inhibited during the production of cheese or during the shelf life of the cheese. In another embodiment, Listeria is of species Listeria monocytogenes or Listeria innocua. In a further embodiment, the cheese is cottage cheese. Methods for making cottage cheese are known in the art and for example mentioned in U.S. Pat. No. 3,298,836 and WO91/00690A1.

The present invention further provides a cheese, such as a cottage cheese or pasta filata cheese, comprising bacteria of the strain Lactobacillus rhamnosus DSM 32092 or a mutant of Lactobacillus rhamnosus DSM 32092, wherein the mutant maintains at least 75%, such as at least 80%, 85%, 90%, 92%, 95%, 98% or 99%, of the anti-Listerial activity of Lactobacillus rhamnosus DSM 32092. In one embodiment, Lactobacillus rhamnosus DSM 32092 is present in the cheese product in a concentration of at least 1×10⁷ cfu/g or at least 1×10⁸ cfu/g, such as at least 5×10⁸ cfu/g or at least 1×10⁹ cfu/g.

The present invention also provides a cheese starter culture characterized in that it comprises Lactobacillus rhamnosus DSM 32092 or a mutant thereof. The culture is preferably present in a frozen, dried or freeze-dried form, e.g. as a Direct Vat Set (DVS) culture. However, as used herein the culture may also be a liquid that is obtained after suspension of the frozen, dried or freeze-dried cell concentrates in a liquid medium such as water or PBS buffer. Where the culture is a suspension, the concentration of viable cells is in the range of 10⁴ to 10¹² cfu (colony forming units) per ml of the composition including at least 10⁴ cfu per ml of the composition, such as at least 10⁵ cfu/ml, e.g. at least 10⁶ cfu/ml, such as at least 10⁷ cfu/ml, e.g. at least 10⁸ cfu/ml, such as at least 10⁹ cfu/ml, e.g. at least 10¹⁰ cfu/ml, such as at least 10¹¹ cfu/ml.

The composition may additionally comprise cryoprotectants, lyoprotectants, antioxidants, nutrients, fillers, flavorants or mixtures thereof. The composition may be in frozen or freeze-dried form. The composition preferably comprises one or more of cryoprotectants, lyoprotectants, antioxidants and/or nutrients, more preferably cryoprotectants, lyoprotectants and/or antioxidants and most preferably cryoprotectants or lyoprotectants, or both. Use of protectants such as croprotectants and lyoprotectantare known to a skilled person in the art. Suitable cryoprotectants or lyoprotectants include mono-, di-, tri- and polysaccharides (such as glucose, mannose, xylose, lactose, sucrose, trehalose, raffinose, maltodextrin, starch and gum arabic (acacia) and the like), polyols (such as erythritol, glycerol, inositol, mannitol, sorbitol, threitol, xylitol and the like), amino acids (such as proline, glutamic acid), complex substances (such as skim milk, peptones, gelatin, yeast extract) and inorganic compounds (such as sodium tripolyphosphate). Suitable antioxidants include ascorbic acid, citric acid and salts thereof, gallates, cysteine, sorbitol, mannitol, maltose. Suitable nutrients include sugars, amino acids, fatty acids, minerals, trace elements, vitamins (such as vitamin B-family, vitamin C). The composition may optionally comprise further substances including fillers (such as lactose, maltodextrin) and/or flavorants.

### Determination of Anti-Listerial Activity

The assay for determining anti-Listerial activity can be performed in a cottage cheese model using the following steps:

Heat-treat skim milk (0.1% fat, 3.6% protein) at 90° C. for 5 minutes. Inoculate 200 ml treated milk simultaneously with 0.029% (w/w) starter culture of Lactococcus lactis subsp. lactis and Streptococcus thermophilus (Fresco 1000NG-10, Chr. Hansen A/S, Denmark) and 1×10⁷ CFU/mL of the strain to be determined.

Ferment the inoculated milk at 35° C. until reaching pH 4.75. Afterwards, place samples in a water bath at 57° C. for 90 minutes. Centrifuge at 500 g for 3 minutes and remove supernatant to obtain a curd. Cool the curd to 12-13° C. and store at 13° C. for later mixing with dressing.

To make the dressing, heat-treat cream (10.5% fat, no added salt) at 90° C. for 5 minutes to ensure low background flora. Inoculate the heat-treated cream with a mix of three L. monocytogenes strains in equal amounts:
mhl210 (obtainable from the Copenhagen University from Department of Veterinary and Animal Sciences, Section for Food Safety and Zoonoses),
ATCC 13932 (obtainable from ATCC) and
DSM 15675 (obtainable from DSMZ).

Purify each strain using Listeria selective PALCAM agar (Oxoid®, Thermo Fisher Scientific, Waltham, Mass.). Take a loop of material using an inoculation loop and transferred to a tube containing 10 mL PALCAM media. Incubate the tube at 30° C. overnight. Transfer 200 μL of the inoculum to 200 mL B-milk (ISO 26323:2009) and grow overnight at 30° C. to acclimate the Listeria strains to milk environment.

Inoculate the dressing with the mixture of Listeria monocytogenes. Mix the dressing with the curd (ratio 60:40% (w/w)) to give a final Listeria concentration of 1×10⁴ CFU/g. Adjust the pH to 5.3 using NaOH to have the same starting pH and viable conditions for L. monocytogenes to grow. Store the sample at 7° C.

Measure the pH once a week and adjust with NaOH the pH to 5.3 again if the pH is below 5.1.

Sample 5 mL of cottage cheese on day 16 and add it to a stomacher bag with 45 mL demineralized water. Stomach the sample until homogenized prepare a dilution row from 10⁻¹ to 10⁻⁶ and plate on Listeria-selective PALCAM agar plates (Oxoid® CM877, Thermo Fisher Scientific, Waltham, Mass.). Incubate the plates at 30° C. for 2-3 days for enumeration. The colonies are grey-green in color with a black halo against the red medium background

According to the Codex Alimentarius, cheeses can be classified using a texture-based classification, established according to the percentage of moisture on a fat-free basis (MFFB). A decrease in MFFB results in a distinction between soft, semisoft, semihard and hard cheeses. Such cheeses are prepared with a ripening step.

The particular invention is especially useful for soft cheese and semisoft cheese as well as fresh cheese.

Soft and Semisoft Cheese Soft and semisoft cheeses represent the riskiest category of cheeses with regard to L. monocytogenes, due to their favorable pH and aw. Soft cheese is manufactured with a relatively short ripening time and has a creamy texture. It can be manufactured from enzymatic or lactic curd. Soft cheeses can be divided into two main categories. On the one hand, mold-ripened soft cheeses have a typical white rind, composed of Penicillium camemberti and/or Geotrichum candidum. Camembert and Brie are well known mold-ripened soft cheeses. On the other hand, smear-ripened soft cheeses, that is washed rind or bacterium-ripened soft cheeses, generally present red rinds. During ripening, they are brushed or washed with salted water which may contain specific starters. The rind is generally composed of Actinobacteria. Pressing is part of the production process of semisoft cheese, but due to a limited ripening time and high water content, it remains creamy and foldable. A wide variety of semisoft cheeses can be found. In European countries, examples include Saint-Paulin and Reblochon. Blue-veined cheeses, containing Penicillium roqueforti in their core, are considered as soft or semisoft cheeses.

There is a great diversity among soft and semisoft cheeses in terms of processing steps. Soft and semisoft cheeses may have a pH ranging from 4.16 to 7.47, and an aw from 0.93 to 0.99. However, the majority of soft and semisoft cheeses present physico-chemical conditions that are favourable for the survival and growth of L. monocytogenes, in terms of both pH and aw.

Smear-ripened soft cheese is more likely to be contaminated with the Listeria, due to the high amount of postprocessing handling, including rind washing and cheese turning. Furthermore, in comparison with the core, cheese rinds are much less acidic, and thus more favourable for the multiplication of the pathogen. For instance, Camembert or Brie rinds can have a pH higher than 7. In addition, pH levels may increase in the rind during the ripening of some soft cheeses. Ripening and storage are thus critical stages.

Examples of soft and semisoft cheeses include Camembert, Brie, Roquefort, Civil, Kopanisti, Munster, Reblochon, Taleggio, cottage cheese, goat cheese, UF-feta, white brined cheese, Mozzarella, Danablu, Stilton, Gorgonzola, Limburger, Tilsit, etc.

### Fresh Cheese

Fresh cheese are curd-style cheeses which do not undergo any ripening. Manufacture generally involves lactic curdling and only a small concentration of rennet. The physico-chemical properties of fresh cheese are generally ideal for the growth of the Listeria-high moisture content (>50%), average pH higher than 6, and relatively low salt content (0.85%). Several large-scale listeriosis outbreaks due to the consumption of fresh cheese have been taken place. One example of fresh cheese includes Queso Fresco, an unripen cheese originated from Latin American countries. Other cheeses that are similar to Queso Fresco include Minas Frescal from Brazil, Burgos from Spain, Panela from Mexico, Quesito from Colombia. A skilled person in the art can readily determine and identify these cheeses for example in accordance with regional regulations, such as Technical Regulations of Cheese Identity and Quality (Regulamento Técnico MERCOSUL de Identidade e Qualidade de Queijos). Other examples include Requeson, Adobera, Burrata, Ricotta, Jben, Kareesh, etc.

### Hard and Semihard Cheese

Hard and semihard cheeses are characterized by a lower water activity compared to fresh, soft and semisoft cheeses. The decrease in aw is obtained by fast curdling, eventual cooking and intensive pressing of the curd, combined with an extended ripening period. The pH of hard cheeses is rather variable, with values ranging from 4.9 to 8.0. Hard cheeses present aw values ranging from 0.91 to 0.97. The lower aw generally creates less unfavorable conditions for survival and growth of L. monocytogenes. Nevertheless, the present invention can still be applied to hard and semihard cheese if contamination during postpasteurization or postprocessing step is likely to occur. Examples of hard and semihard cheeses include Cheddar, Red Leicester, American cheese, Gouda, Edam, Emmental, an Italian cheese such as Parmesan, Parmigiano, Regiano, Grana Padano, Provolone, Pecorino, or Romano.

To prepare products in accordance with the present application, milk or milk substrate can be provided as starting material. The method of the present application is characterized in that the milk or milk substrate is inoculated with the L. rhamnosus DSM 32092 or a mutant of Lactobacillus rhamnosus DSM 32092 with at least 75% of the anti-Listerial activity.

Inoculation of DSM 32092 can be carried out in a concentration of at least 1×10⁶ CFU/ml or at least 1×10⁷ CFU/ml or at least 1×10⁸ CFU/ml in the starting material. In other embodiments, inoculation may be in a concentration range of 1×10⁶ to 1×10⁸ CFU/mL, preferably in a concentration range of 5×10⁶ to 1×10⁸ CFU/mL, most preferably in a concentration of 1×10⁷ to 1×10⁸ CFU/mL.

In a further embodiment, the milk is inoculated with a starter culture and the L. rhamnosus DSM 32092 or a mutant of Lactobacillus rhamnosus DSM 32092, wherein the mutant maintains at least 85% of the anti-Listerial activity of Lactobacillus rhamnosus DSM 32092. As used herein, microorganisms used for inoculation is collectively referred to as culture.

A skilled person in the art will also include the inoculation of primary starter culture, depending on the product to be made. In some embodiment, the primary starter culture comprises Lactococcus lactis subsp. lactis and Streptococcus thermophilus, including a starter culture consisting of Lactococcus lactis subsp. lactis and Streptococcus thermophilus.

After inoculation, the milk or milk substrate is subject to suitable conditions for fermentation. Fermentation causes a decrease in pH and allows for flavors to develop. It is within the skills of ordinary practitioners to determine the type starter culture and amounts to be used, as well as fermentation parameters, depending on the desired dairy product to be produced.

Preferably, fermentation is carried out at 30 to 40° C., such as at 32 to 39° C., such as 34 to 38° C., as this is a suitable temperature for DSM 32092. Fermentation time can be from 3 hours to 20 hours, such as 4-15 hours, such as 5-10 hours.

After fermentation, coagulants are used to coagulate the milk or milk substrate. A coagulant is typically an enzyme that is able to separate the milk into solid and liquid components. The resulting solid component is known as curd while the liquid component is called whey.

Methods of coagulation include contacting the milk with chymosin originating from the abomasum, of a calf or with a commercially available coagulant such as chymosin (EC 3.4.23.4). Coagulants are well known in the art and commercially available, for example CHY-MAX® or CHY-MAX® M (Chr. Hansen A/S, Denmark).

Other useful coagulants may be the mucorpepsin microbial coagulant produced by fermentation using the fungus Rhizomucor miehei, which are commercially available as Hannilase® or Microlant® (Chr. Hansen A/S, Denmark).

After adding the coagulant and subjecting the milk base to a suitable condition, the coagulation process begins and continues for a period of time. A person of ordinary skill in the art can readily select suitable process conditions, such as the cheese type, temperature, oxygen, addition of carbohydrates, amount and characteristics of milk base and the processing time. This process may take from two, three, four, five, six hours or longer. Preferably, coagulation is carried out at 30 to 40° C., such as at 32 to 39° C., such as 34 to 38° C.

In one aspect, the present invention relates to a method for inhibiting Listeria in cheese, comprising the following steps:
providing milk or milk substrate;
inoculating the milk or milk substrate with a culture comprising Lactobacillus rhamnosus DSM 32092 and fermenting the milk or milk substrate,
forming a curd,
using the curd to produce cheese.

Curd formation can take place due to acidification from culture fermentation or by addition of coagulant(s). In one aspect, the present invention comprises the following steps:
providing milk or milk substrate;
inoculating the milk or milk substrate with a culture comprising Lactobacillus rhamnosus DSM 32092 and fermenting the milk or milk substrate,
forming a curd by adding a coagulant to the milk or milk substrate, and
using the curd to produce cheese.

In one embodiment, the method comprises the following steps:
providing milk or milk substrate,
inoculating the milk or milk substrate with a culture comprising Lactobacillus rhamnosus DSM 32092 and cheese or yogurt starter culture, and
obtaining cheese or yoghurt products.

In another embodiment, the method comprises the following steps:
providing milk and pasteurizing the milk,
inoculating the milk with a culture as described in the present application,
heating the milk to a renneting temperature and optionally adding coagulant to obtain a curd,
cutting the curd,
removing whey,
molding the curd,
cooling and brining the curd, and
packing the cheese.

Inoculation of DSM 32092 in the milk or milk substrate can be at least 1×10⁶, such as at least 1×10⁷, such as at least 1×10⁸, for example in a concentration range of 1×10⁶ to 1×10⁸ CFU/mL, preferably in a concentration range of 5×10⁶ to 1×10⁸ CFU/mL, most preferably in a concentration of 1×10⁷ to 1×10⁸ CFU/mL.

In one aspect, the present invention provides use of bacteria of the strain Lactobacillus rhamnosus DSM 32092 or a mutant of Lactobacillus rhamnosus DSM 32092 thereof for the inhibition of Listeria. A skilled person is able to use the bacteria as taught throughout the application. In one embodiment, Listeria is inhibited during the production of cheese and/or during the shelf life of the cheese, preferably fresh cheese, soft cheese, or semisoft cheese. The cheese may be cottage cheese, white brined cheese, rindless soft cheese, white mold soft cheese, smear-ripened soft cheese, blue-veined soft cheese and pasta filata cheese.

In some embodiments, DSM 32092 is inoculated in a concentration of at least 1×10⁶′ such as at least 1×10⁷, such as at least 1×10⁸, for example in a concentration range of 1×10⁶ to 1×10⁸ CFU/mL, preferably in a concentration range of 5×10⁶ to 1×10⁸ CFU/mL, most preferably in a concentration of 1×10⁷ to 1×10⁸ CFU/mL. DSM 32092 can be used with a starter culture comprising Lactobacillus spp., Streptococcus spp., Leuconostoc spp. and/or Lactococcus spp. or combinations thereof.

Soft cheeses can be raw milk soft cheese or pasteurized soft cheese. Examples of cheeses include Camembert, Brie, Roquefort, Civil, Kopanisti, Munster, Reblochon, Taleggio, cottage cheese, goat cheese, UF-feta, white brined cheese, Mozzarella, Danablu, Stilton, Gorgonzola, Limburger, Tilsit, etc.

In one embodiment the cheese is produced using a starter culture which does not comprise bacteria of the species Lactobacillus fermentum.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### Deposit and Expert Solution

The applicant requests that a sample of the deposited microorganisms stated below may only be made available to an expert, subject to available provisions governed by Industrial Property Offices of States Party to the Budapest Treaty, until the date on which the patent is granted.

The applicant deposited the Lactobacillus rhamnosus DSM 32092 on 2015-07-16 at Leibniz Institute DSMZ—German Collection of Microorganisms and Cell Cultures, Inhoffenstr. 7B, D-38124 Braunschweig and received the accession No.: DSM 32092.

### EXAMPLES

### Example 1 Analysis of the Inhibitory Effect of Lactobacillus rhamnosus DSM 32092 on the Growth of Listeria monocytogenes in Cottage Cheese Model Compared with LMG 18030, LMG 18025, LMG 18020 and CNCM I-4316

A cottage cheese model was provided for a quantitative examination of the inhibitory effect of Lactobacillus rhamnosus in dairy product. This mimics the condition of warm-filled cottage cheese made in the USA. Inhibitory effect of DSM 32092 was compared with L. rhamnosus described in WO2017/050980 (LMG 18030, LMG 18025, LMG 18020 and CNCM I-4316). Regular skimmed milk (0.1% fat, 3.6% protein) was heat-treated at 90° C. for 5 minutes to ensure a low background flora and transferred to 200 mL bottles. Then, 200 ml milk were inoculated with 0.029% (w/w) starter culture of Lactococcus lactis subsp. lactis and Streptococcus thermophilus (Fresco1000NG-10, Chr. Hansen A/S, Denmark) and 1×10⁷ CFU/mL L. rhamnosus as shown in Table 1. A sample only with Fresco 1000NG-10 was included as control.

TABLE 1
Experimental design

| Sample | Primary starter culture | L. rhamnosus culture |
|---|---|---|
| Control | Lactococcus lactis subsp. lactis | None |
| | Streptococcus thermophilus | |
| 1 | Lactococcus lactis subsp. lactis | DSM 32092 |
| | Streptococcus thermophilus | |
| 2 | Lactococcus lactis subsp. lactis | LMG 18030 |
| | Streptococcus thermophilus | |
| 3 | Lactococcus lactis subsp. lactis | LMG 18025 |
| | Streptococcus thermophilus | |
| 4 | Lactococcus lactis subsp. lactis | LMG 18020 |
| | Streptococcus thermophilus | |
| 5 | Lactococcus lactis subsp. lactis | CNCM I-4316 |
| | Streptococcus thermophilus | |

Each bottle was fermented at 35° C. until reaching pH 4.75. The bottles were heat-treated for 90 minutes in a water bath at 57° C. to simulate cottage cheese scalding. The bottles were centrifuged at 500 g for 3 minutes and the supernatant was removed and a curd was obtained. The curd was cooled to 12-13° C. and stored at 13° C. for later mixing with the dressing.

To make the dressing, 9% and 38% fat cream were mixed to obtain a fat level of about 10.5%. The dressing was heat-treated at 90° C. for 5 minutes.

The dressing for the cottage cheese model was inoculated with a mix of three L. monocytogenes strains in equal amounts:
mhl210 (obtainable from the Copenhagen University from Department of Veterinary and Animal Sciences, Section for Food Safety and Zoonoses),
ATCC 13932 (obtainable from ATCC) and
DSM 15675 (obtainable from DSMZ).

Each strain had been purified using Listeria selective PALCAM agar (Oxoid). A loop of material is taken using an inoculation loop and transferred to a tube containing 10 mL PALCAM media. The tube was incubated at 30° C. overnight. 200 μL of the inoculum was transferred to 200 mL B-milk (ISO 26323:2009) and grown overnight at 30° C. to acclimate the Listeria strains to milk environment.

The mixture of Listeria monocytogenes was inoculated in the cottage cheese dressing, and the dressing was mixed 60:40% (w/w) with the curd, giving a Listeria concentration of 1×10⁴ CFU/g. The pH was adjusted to 5.3 using NaOH. The samples were stored at 7° C.

The pH was measured once a week and adjusted with NaOH the pH to 5.3 again if the pH is below 5.1.

One mL of each cottage cheese was sampled on day 2, 7, 12, 16 and 20 of the experiment, stomached to ensure a homogenous sample, diluted, and plated on Listeria-selective PALCAM agar plates (van Netten P. et al. (1989) Int. J. Food Microbiol. 8. (4) 299-316). The plates were incubated at 30° C. for 2-3 days and Listeria was enumerated.

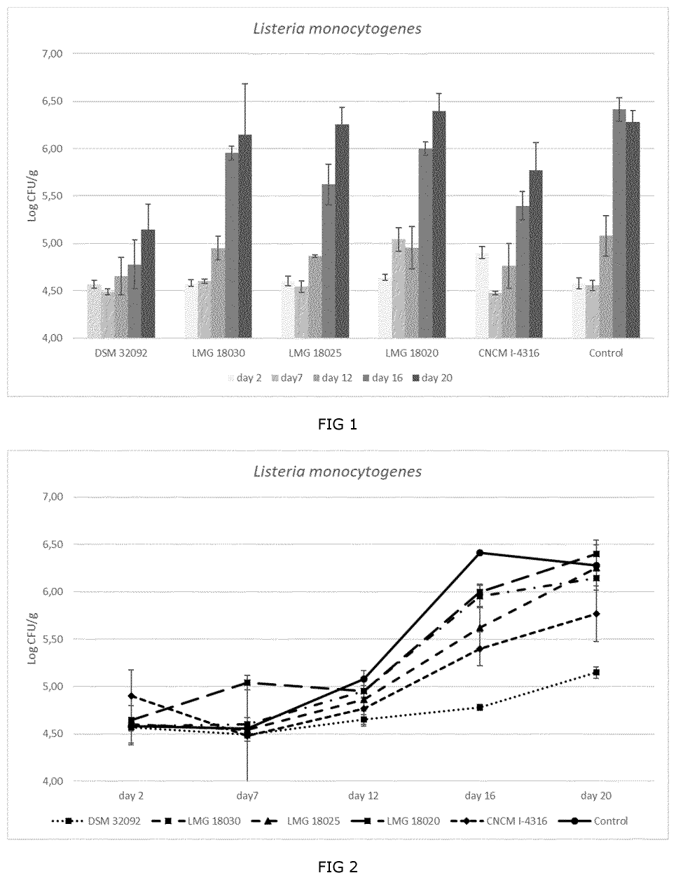

FIGS. 1 and 2 show the Listeria level (Log CFU/g) in the tested samples. Lactobacillus rhamnosus DSM 32092 shows significantly higher inhibition than LMG 18030, LMG 18025, LMG 18020 and CNCM I-4316. The effect was observed on all of the samples and still significant after 20 days.

### Example 2 Inhibition of Listeria in Cottage Cheese

This example is a laboratory-based study that measures the growth of L. monocytogenes in artificially contaminated food prepared and stored under conditions that might be expected to occur throughout the cold chain, including storage conditions between production and consumption. The test period started the day of contamination and finishes at the end of the shelf-life. The growth potential is the difference between the log 10 cfu/g at the end of the test and the log 10 cfu/g at the beginning of the test.

### Preparation of Curd

A control curd and a test curd were prepared. For both curds, commercial skimmed milk with about 0.05% fat and 3.2% protein was pasteurized at 73° C. for 15 seconds and added to the cheese vat and brought to a temperature of 36° C. Commercial starter culture containing Lactococcus lactis subsp. lactis and Streptococcus thermophilus (Fresco1000NG-10, Chr. Hansen A/S, Denmark) was added to the tempered cheese milk (100 U/380 L). For the test curd, DSM 32092 was inoculated at 2×10⁷ CFU/ml.

The vats were incubated at 36° C. for approximately 5 hours until the pH had reached 4.65. The coagulum was cut manually with 6.35 mm cottage cheese knives to form cubes, first cut horizontal then vertical. The resulting curd/whey mixture was incubated with temperature held at 36° C. for 20 minutes in the vat to heal the curd. The curd/whey mixture was then heated in a two stage process: first heated to 43° C. for 1 hour with slow continuous stirring (8 rpm) and then heated to 57° C. for 30 minutes with slow continuous stirring (8 rpm). Following the second heat treatment, the whey was drained from the curd for a 15 minute period. The drained curd was washed in the vat manually with cold potable water (13° C.) added to completely cover the curd and under continuous agitation (8 rpm) then drained after 5 minutes. A second wash was repeated, again using cold potable water (2° C.) under continuous stirring (8 rpm) for 5 minutes then drained. The two-stage wash cooled the curd to approximately 7° C. for a period of 30 minutes. The curd was then allowed to fully drain in the cheese vat for 20 minutes before removal from the cheese vat.

### Preparation of Dressing

By weight, 78% raw skim milk (0.05% fat), 19.8% raw cream (40% fat) and 2.2% salt (NaCL) were blended. After blending the mixture was homogenized (1^{st} stage 1500 psi/2^{nd} stage 500 psi; total 2000 psi) and then pasteurized (HTST) at 87.5° C. for 25-30 seconds. The pasteurized dressing was then cooled to 3° C. The prepared dressing contained 8% fat and 2.29% salt.

### Preparation of Cottage Cheese

Control cottage cheese and test cottage cheese were prepared by combining equal weights of dressing and control curd or test curd.

The target pH of the equilibrated cottage cheese mixture was 5.20-5.25 (measured as direct pH on a blended sample at room temperature). The pH was adjusted with NaOH/HCl as necessary to achieve target. Natamycin, a mold inhibitor with no effect on bacteria, was added to the control cottage cheese to prevent mold growth that could raise pH and accelerate L. monocytogenes growth. The final fat level of the product was 4%.

### Proximate Analysis

Proximate analysis was completed in triplicate on control curd, test curd, dressing, as well as control cottage cheese and test cottage cheese. The following parameters were determined:
moisture (5 h, 100° C., vacuum oven method in accordance with Official Methods of ACOC International, AOAC Method 950.46)
pH (direct measurement on homogenized sample) using Orion Star A111 pH meter and Orion 8104 combination electrode, Thermo Fisher Scientific, Waltham, Mass.)
NaCl (measured as % Cl⁻, AgNO₃ potentiometric titration, G20 Compact Titrator, Mettler Toledo, Columbus, Ohio)
Water activity (Decagon AquaLab TE4 or Series 3 water activity meter, Pullman, Wash.).

Details for proximate analysis results are shown in Table 2.

TABLE 2

| Formulation | % Moisture | Water Activity | pH | % NaCl |
|---|---|---|---|---|
| Control Curd | 81.44 | 0.997 | 4.49 | 0.07 |
| | 81.21 | 1.000 | 4.49 | 0.06 |
| | 81.49 | 0.996 | 4.49 | 0.06 |
| Average ± SD | 81.38 ± 0.15 | 0.998 ± 0.002 | 4.49 ± 0.00 | 0.06 ± 0.01 |
| Test Curd | 80.34 | 0.996 | 4.51 | 0.05 |
| | 80.18 | 0.999 | 4.52 | 0.06 |
| | 80.06 | 0.999 | 4.52 | 0.05 |
| Average ± SD | 80.19 ± 0.14 | 0.998 ± 0.002 | 4.52 ± 0.01 | 0.05 ± 0.00 |
| Dressing | 82.84 | 0.983 | 6.63 | 2.19 |
| | 82.86 | 0.983 | 6.65 | 2.16 |
| | 82.82 | 0.983 | 6.65 | 2.13 |
| Average ± SD | 82.84 ± 0.02 | 0.983 ± 0.000 | 6.64 ± 0.01 | 2.16 ± 0.03 |
| Control Cheese | 83.12 | 0.988 | 5.10 | 1.11 |
| | 83.34 | 0.987 | 5.08 | 1.10 |
| | 83.11 | 0.986 | 5.08 | 1.15 |
| Average ± SD | 83.19 ± 0.13 | 0.987 ± 0.001 | 5.09 ± 0.01 | 1.08 ± 0.08 |
| Test Cheese | 82.43 | 0.990 | 5.13 | 1.12 |
| | 82.69 | 0.989 | 5.14 | 1.11 |
| | 82.72 | 0.994 | 5.16 | 1.07 |
| Average ± SD | 82.61 ± 0.16 | 0.991 ± 0.003 | 5.14 ± 0.02 | 1.10 ± 0.03 |

L. monocytogenes Inoculum

To account for variation in growth and survival among strains of L. monocytogenes, a challenge test was conducted with a pool of strains. A pool of 6-strain mixture of acid-adapted L. monocytogenes FSL R2-501, FSL R2-500, LM101, LM310, LM301, LM108 was used as target microorganisms.

TABLE 3

| Strain | | | |
|---|---|---|---|
| designa- | Sero- | | Culture |
| tion | type | Source | Collection |
| FSL | 4b | Hispanic-style cheese isolate, | ILSI Cornell |
| R.2-501 | | associated with NC outbreak | |
| FSL | 4b | clinical isolate, associated with NC | ILSI Cornell |
| R.2-500 | | Hispanic-style cheese outbreak | |
| LM101 | 4b | hard salami isolate | UW-Madison Food |
| | | | Research Institute |
| LM310 | 4b | goat cheese isolate associated | UW-Madison Food |
| | | with illness | Research Institute |
| LM301 | 1/2a | heat-treated Cheddar cheese | UW-Madison Food |
| | | isolate | Research Institute |
| LM108 | 1/2b | hard salami isolate | UW-Madison Food |
| | | | Research Institute |

Stocks of these strains were maintained in ceramic beads (CRYO/M; Copan Diagnostics Inc., Murrieta, Calif.) stored at −80° C. For inoculum preparation, each individual strain bead was cultured in 10 ml of fresh trypticase soy broth (TSB; Becton, Dickinson and Company, Sparks, Md., USA) at 37° C. for 20 to 24 h. The freshly grown culture (0.1 ml) was further transferred into 10 ml of fresh TSB supplemented with 1% glucose and incubated at 37° C. for 18-22 h until stationary phase and to induce acid tolerance (Buchanan, Robert L., and Sharon G. Edelson. “pH-dependent stationary-phase acid resistance response of enterohemorrhagic Escherichia coli in the presence of various acidulants.” Journal of food protection 62.3 (1999): 211-218). Cells were harvested by centrifugation (4,000×g, 20 min) and suspended in 4.5 ml 0.1% buffered peptone water (BPW, pH 7.1±0.1). Strains were mixed in approximately equal concentrations (volume used based on population estimates using optical density) and the inocula diluted in BPW to deliver approximately 3.5-log CFU/g cottage cheese using a 0.5% liquid inoculum. Populations of viable cells of the six-strain mixture and of individual strains were verified by plating serial dilutions on Modified Oxford agar (MOX) (Hitchens, D., and K. Jinneman. “BAM: detection and enumeration of Listeria monocytogenes, 2011.” (2012)).

For both the control cheese and the test cheese, 5000 g cottage cheese (about 13° C.) was measured into a sterilized stainless steel bin, and pH adjusted to 5.25±0.05 using 10 N HCl. For the control cottage cheese, 0.002% (w/w) natamycin powder was added by dusting over the surface of the finished product and mixing for 3 minutes. Then, both cheeses were inoculated with 2.5 ml (0.5% v/w) prepared inoculum by dripping inoculum over the surface with a pipette and gently hand-stirring with a sterile stainless steel spatula for approximately 5 minutes.

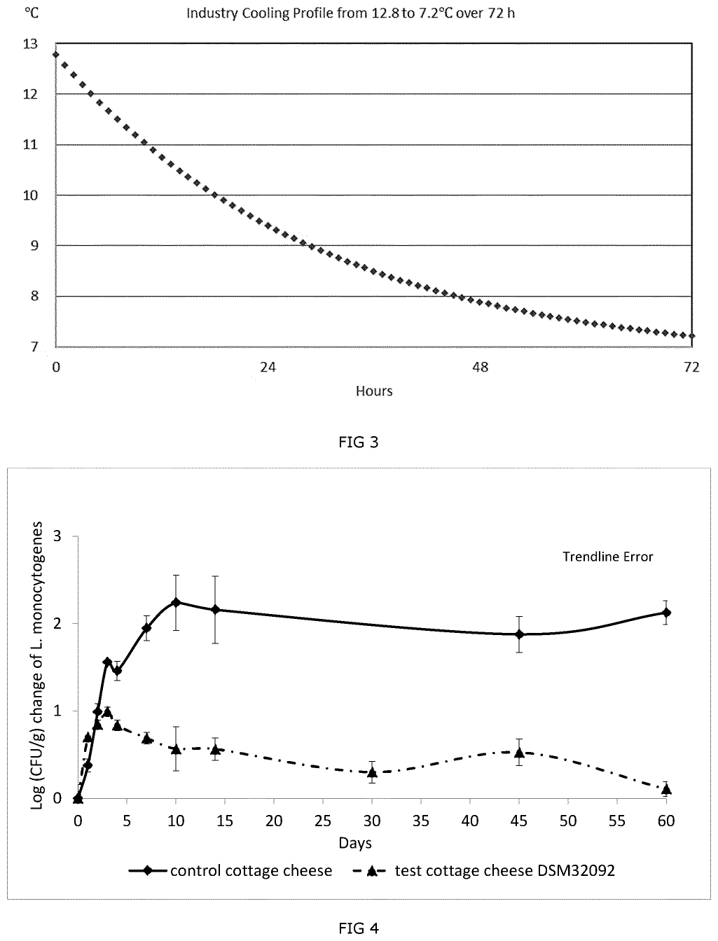

Inoculated cottage cheese was dispensed into 60 ml screw-top jars and cooled by industry cooling practice (from 12.8° C. to 7.2° C. for 72 hours) in a programmable incubator (Model 7901-25-2, CARON products, Marietta Ohio), and then stored at 7.2° C. for the remainder of the 60 days storage testing. The industry cooling profile was developed by the Center for Food Safety and Applied Nutrition (CFSAN) in July 2005 from data supplied by the International Dairy Foods Association (IDFA) for a representative variety of product sizes and cooling rates (see cooling curve in FIG. 3 below). The internal temperature for each product type was monitored with a thermocouple (type K probe) and using an I-button temperature probe (iButtons; DS1922T-F5, Maxim Integrated, San Jose, Calif.) inserted into one cup for each cooling scenario. Thermocouples were calibrated against a factory-calibrated mercury-filled thermometer (FisherBrand, factory-calibrated to meet the requirements of ISO/EC Guide 25, ANSI/NCSL 2540-1-1994, ISO 9000/QS 9000 Series of Quality Standards, and MIL STD 45662A).

### Sampling

For each sampling interval (0, 1, 2, 3, 4, 7, 10, and 14 days' storage), triplicate samples for control and test cheese were removed from incubation and assayed by aseptically removing a 25-g sample, diluting with an equal volume 0.1% peptone water, homogenizing with a Stomacher, and then plating serial dilutions on Modified Oxford Agar (MOX). The samples were also assayed at different 30, 45, and 60 days to determine if the same growth trends would persist through long-term storage. pH was measured on remaining blended, undiluted samples at each sampling time. Listeria monocytogenes was enumerated (cfu/g) on Modified Oxford Agar (MOX; 35-37° C., 48 h). pH was directly measured using Orion Star A111 pH meter and Orion 8104 combination electrode (Thermo Fisher Scientific, Waltham, Mass.). Average log change in Listeria monocytogenes populations over 60 days was calculated.

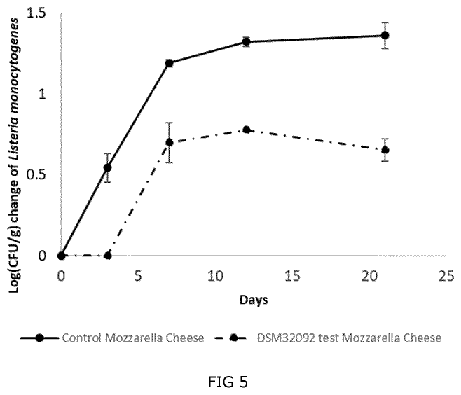

Table 4 shows population of Listeria monocytogenes (LM) and pH change in the control and tested cottage cheese. Growth change is also depicted in FIG. 4.

This example demonstrates that growth inhibition of Listeria monocytogenes during long term storage can be achieved using DSM 32092.

TABLE 4
Growth of Listeria monocytogenes (LM) and pH in the test and control cheese

| | Control Cottage Cheese | | | Test Cottage Cheese | | |
|---|---|---|---|---|---|---|
| | | Average LM | | | Average LM | |
| | | log (CFU/g) | | | log (CFU/g) | |
| | LM log | change from | | LM log | change from | |
| Time point | (CFU/g) | 0-time | pH | (CFU/g) | 0-time | pH |
| Day 0 | 4.15 | | 5.18 | 3.90 | | 5.22 |
| | 3.86 | | 5.22 | 3.96 | | 5.18 |
| | 4.11 | | 5.24 | 3.88 | | 5.20 |
| Average ± SD | 4.04 ± 0.16 | | | 3.91 ± 0.04 | | |
| Day 1 | 4.33 | | 5.20 | 4.60 | | 5.20 |
| | 4.46 | | 5.17 | 4.63 | | 5.24 |
| | 4.46 | | 5.20 | 4.61 | | 5.22 |
| Average ± SD | 4.42 ± 0.08 | +0.38 | | 4.61 ± 0.02 | +0.70 | |
| Day 2 | 4.97 | | 5.01 | 4.76 | | 5.07 |
| | 5.14 | | 5.03 | 4.76 | | 5.07 |
| | 4.98 | | 5.02 | 4.76 | | 5.10 |
| Average ± SD | 5.03 ± 0.10 | +0.99 | | 4.76 ± 0.00 | +0.85 | |
| Day 3 | 5.61 | | 4.98 | 4.93 | | 5.04 |
| | 5.60 | | 4.97 | 4.94 | | 5.07 |
| | 5.59 | | 4.99 | 4.85 | | 5.05 |
| Average ± SD | 5.60 ± 0.01 | +1.56 | | 4.91 ± 0.05 | +0.99 | |
| Day 4 | 5.48 | | 4.91 | 4.79 | | 4.98 |
| | 5.62 | | 4.94 | 4.78 | | 4.99 |
| | 5.40 | | 5.04 | 4.68 | | 5.03 |
| Average ± SD | 5.50 ± 0.11 | +1.46 | | 4.75 ± 0.06 | +0.84 | |
| Day 7 | 6.15 | | 4.90 | 4.56 | | 4.89 |
| | 5.89 | | 4.88 | 4.57 | | 4.91 |
| | 5.92 | | 4.81 | 4.68 | | 4.92 |
| Average ± SD | 5.99 ± 0.14 | +1.95 | | 4.60 ± 0.07 | +0.69 | |
| Day 10 | 6.64 | | 4.87 | 4.60 | | 4.84 |
| | 6.16 | | 4.86 | 4.65 | | 4.88 |
| | 6.04 | | 4.82 | 4.19 | | 4.86 |
| Average ± SD | 6.28 ± 0.32 | +2.24 | | 4.48 ± 0.25 | +0.57 | |
| Day 14 | 6.64 | | 4.84 | 4.44 | | 4.81 |
| | 6.03 | | 4.84 | 4.37 | | 4.86 |
| | 5.93 | | 4.85 | 4.62 | | 4.86 |
| Average ± SD | 6.20 ± 0.38 | +2.16 | | 4.48 ± 0.13 | +0.56 | |
| Day 30 | | | | 4.28 | | 4.65 |
| | | Not tested | | 4.29 | | 4.70 |
| | | | | 4.07 | | 4.68 |
| Average ± SD | | | | 4.21 ± 0.12 | +0.30 | |
| Day 45 | 6.06 | | 4.77 | 4.32 | | 4.55 |
| | 5.68 | | 4.74 | 4.39 | | 4.59 |
| | 6.00 | | 4.77 | 4.61 | | 4.57 |
| Average ± SD | 5.92 ± 0.21 | +1.88 | | 4.44 ± 0.15 | +0.53 | |
| Day 60 | 6.22 | | 4.74 | 4.10 | | 4.49 |
| | 6.01 | | 4.76 | 4.03 | | 4.49 |
| | 6.27 | | 4.76 | 3.93 | | 4.48 |
| Average ± SD | 6.17 ± 0.14 | +2.13 | | 4.02 ± 0.09 | +0.11 | |

### Example 3 Preparation of White Brined Cheese with DSM 32092

Pasteurized milk (72° C. for 15 sec) is standardized (2.9% fat, 3.55% protein) and preheated to 35° C. A commercial starter culture containing Lactobacillus delbrueckii subsp. bulgaricus and Streptococcus thermophilus (F-DVS WhiteClassic 200 available from Chr. Hansen A/S Denmark) is inoculated at 0.0075% (v/w) into buckets containing 10 L milk. One bucket is inoculated with Lb. rhamnosus DSM 32092 in total concentration of 2×10⁷ CFU/g. The buckets are incubated at 35±1° C. Rennet (200 IMCU bovine chymosin CHY-MAX, Chr. Hansen A/S Denmark) is added after 30 minutes in a concentration 25 mL per 100 kg milk. Agitation is continued for up to 4 min after rennet addition. CaCl₂ is added to the milk at a rate 6 g CaCl₂ per 100 kg milk. After 90 minutes of coagulation the coagulum is cut (15 mm between the strings). The curd is stirred for 40 minutes by hand paddle. After stirring the whey is drained. When the pH is below 5.9 the cheese curd is transferred into molds and left for drainage. The molds are turned four times within the next 3 hours. After the last turn the pH is measured (pH between 4.8-5.0). The cheese is then stored at 26° C. overnight. On the next day the cheese is added to brine with 8% NaCl (w/w), 0.75% CaCl2 and pH 4.6 (adjusted with lactic acid) and stored at room temperature until the pH reached 4.6-4.7.

### Example 4 Preparation of Ultra-Filtrated (UF) White Brined Cheese with DSM 32092

Ultrafiltration-retentate is heated to 32° C. 1° C. A commercial starter culture containing Lactococcus lactis subsp. lactis, Lactococcus lactis subsp. cremoris, Lactobacillus delbrueckii subsp. bulgaricus and Streptococcus thermophilus (F-DVS WhiteDaily 82 available from Chr. Hansen A/S Denmark) is inoculated at 10 U/100 L retentate (v/w). One bucket is inoculated with Lb. rhamnosus DSM 32092 in total concentration of 2×10⁷ CFU/g. The buckets are incubated at 32±1° C. Rennet (HANNILASE® XP 200, Chr. Hansen A/S Denmark) is added after 30 minutes of fermentation in a concentration of 7000 IMCU/100 L retentate (v/w). Agitation is continued for up to 2 min after rennet addition. After 30 minutes of coagulation a parchment paper is added on top of the coagulum and 2% NaCl (w/w) is added on top of the parchment paper. Fermentation is continued at 32° C. until the pH reached 4.6-4.7.

### Example 5 Preparation of Rindless Soft Cheese with DSM 32092

Pasteurized milk (80° C. for 30 sec) is standardized (3.4% fat and 3.3% protein) and heated to 35° C. 1° C. CaCl₂ is added in a concentration of 40 g/100 L milk (v/v). A commercial starter culture containing Streptococcus thermophilus (F-DVS SOFT CREMOSO 1-04 available from Chr. Hansen A/S Denmark) is inoculated at 5 U/100 L (v/v). One vat is inoculated with Lb. rhamnosus DSM 32092 in total concentration of 2×10⁷ CFU/g (v/v). The vats are matured at 35±1° C. for 10 minutes before addition of rennet (chymosin, CHY-MAX® M, Chr. Hansen A/S Denmark) in a concentration 5000 IMCU/100 L milk (v/v). Agitation is continued for up to 4 min after rennet addition. After 20 minutes of coagulation the coagulum is cut (1×1×1 cm) and the curd is resting for 5 minutes before initiating stirring and heating at 39° C. 1° C. for 15 minutes. The curd is stirred in total for 45 minutes and transferred into molds. The molds are turned 3 times within 2.5 hours. Cheeses are kept above 35° C. during draining until pH 5.2-5.3 is reached. The cheeses are brined in cold brine at 4° C. 1° C. for 35 minutes. After brining the cheeses are dried at 12° C. 1° C. for 16 hours before packaging.

### Example 6 Preparation of White Mold Cheese with DSM 32092

Pasteurized milk (80° C. for 30 sec) is standardized (6.1% fat and 3.7% protein) and heated to 41° C. 1° C. CaCl₂ is added in a concentration of 40 g/100 L milk (v/v). A commercial starter culture containing Streptococcus thermophilus (F-DVS WhiteStamp 1000 available from Chr. Hansen A/S Denmark) is inoculated at 10 U/100 L (v/v). Commercial ripening cultures containing Geotrichum candidum (FD-DVS SWING GEO CH, available from Chr. Hansen A/S Denmark) is inoculated at 1 U/1000 L milk (v/v), and Penicillium candidum (FD-DVS SWING PCA-3, available from Chr. Hansen A/S Denmark) is inoculated at 2 U/1000 L milk (v/v). One vat is inoculated with Lb. rhamnosus DSM 32092 in total concentration of 2×10⁷ CFU/g (v/v). The vats are matured at 41±1° C. for 40 minutes before addition of rennet (chymosin, CHY-MAX® Special, Chr. Hansen A/S Denmark) in a concentration 5000 IMCU/100 L milk (v/v). Agitation is continued for up to 4 min after rennet addition. After 35 minutes of coagulation the coagulum is cut (2×1×1 cm) and the curd is stirred very gentle 1-3 minutes and then rested in the vat for 25 minutes until molding. The curd is transferred into molds. The molds are turned 3 times and temperature decreased to 30±1° C. within 4 hours. Then the temperature is decreased to 17-18±1° C. within 16 hours until pH reached 5.15-5.05. The cheeses are brined at 12±1° C. for 50 minutes and put for drying at 16° C. for 1 day, and the temperature decreases from 14±1° C. to 10±1° C. in 9 days at 98% relative humidity. The cheeses are then stored for 8 hours at 4±1° C. before packaging.

### Example 7 Inhibition of Listeria in Pasta Filata Cheese

### Preparation of Mozzarella Cheese

Samples of “control Mozzarella cheese” and “DSM32092 test Mozzarella cheese” were prepared according to the following. Pasteurized whole milk (72° C. for 15 sec) with 3.5% fat and 3.4% protein was heated to a temperature of 32° C. Commercial starter culture containing Streptococcus thermophilus (F-DVS 450 available from Chr. Hansen A/S, Denmark) was inoculated at 50 U/750 L to the tempered cheese milk 32±1° C. in two buckets. One bucket was inoculated with Lb. rhamnosus DSM 32092 in total concentration of 2×10⁷ CFU/g. Rennet (1000 IMCU bovine chymosin CHY-MAX Supreme 1000, Chr. Hansen A/S, Denmark) was added to both buckets in a concentration 50 IMCU/L milk, followed by a short time of stirring for distribution purpose. The buckets were incubated at 32±1° C. for 1 hour before the curd was cut. The curd was left resting at 32±1° C. for 1 hour. The curd was stirred at low rate for 1 min and left for resting for 15 min before repeating. The process was continued until reaching pH 5.3. The cheese curd was stretched, by lowering the curd into maximum 60° C. hot water and obtained a smooth and elastic fibrous protein structure with parallel strings. The cheese was shaped in ball-shapes, packed and stored at 5° C. for 4 days before inoculating with Listeria and performing analysis.

### Proximate Analysis

Proximate analysis on cheese composition; percentage fat, milkfat in dry matter, dry matter and moisture (FoodScan Lab, FOSS, Hillerød, Denmark) was completed on control and DSM32092 test cheese.

Details for proximate analysis results are shown in Table 5.

TABLE 5

| | | % Milkfat | | |
|---|---|---|---|---|
| | | in dry | % Dry | |
| Formulation | % Fat | matter | matter | % Moisture |
| Control Mozzarella | 22.79 | 48.51 | 46.98 | 53.02 |
| Cheese | 22.69 | 48.18 | 47.10 | 52.90 |
| Average ± SD | 22.7 ± 0.1 | 48.35 ± 0.2 | 47.0 ± 0.1 | 53.0 ± 0.1 |
| DSM32092 Test | 22.16 | 47.89 | 47.89 | 53.73 |
| Mozzarella | 22.09 | 47.78 | 47.78 | 53.77 |
| Cheese | | | | |
| Average ± SD | 22.1 ± 0.0 | 47.8 ± 0.1 | 46.3 ± 0.0 | 53.8 ± 0.0 |

L. monocytogenes Inoculum

To account for variation in growth and survival among strains of L. monocytogenes, a challenge test was conducted with a pool of strains. A pool of 3-strain mixture of acid-adapted L. monocytogenes, mhl210, ATCC 13932 and DSM 15675, was used as target microorganisms.

TABLE 6

| Strain designation | Serotype | Source |
|---|---|---|
| mhl210 | 4b | |
| ATCC 13932 | 4b | |
| DSM 15675 | 4b | Isolated from soft cheese |

To obtain ampoules with glycerol stocks containing acid-adapted L. monocytogenes strains (mhl210, ATCC 13932 and DSM 15675), each individual strain was cultured in fresh trypticase soy broth (TSB) from a single colony to early stationary phase at 37° C. for 20 to 24 h. The freshly grown culture was further transferred, 100-fold diluted, into fresh TSB supplemented with 1% glucose and grown at 37° C. for 18-22 h until stationary phase, to induce acid tolerance (Buchanan, Robert L., and Sharon G. Edelson. “pH-dependent stationary-phase acid resistance response of enterohemorrhagic Escherichia coli in the presence of various acidulants.” Journal of food protection 62.3 (1999): 211-218). The TSB+1% Glu L. monocytogenes grown cultures were mixed in equal volumes, frozen, and a CFU count was performed after 24-hrs of freezing to calculate the cell concentration of the stocks. Prior to inoculation on the Mozzarella cheese, 2 mL of a stock ampoule was dissolved in peptone water and used to inoculate the various Mozzarella cheeses to establish the indicated CFU counts.

The Mozzarella cheeses were cut into slices weighing 25 g±2 g and placed in petri dishes. The slices were inoculated on the surface with 20 ul prepared inoculum by dripping inoculum over the surface with a pipette. Inoculated samples were stored at 7° C. for the remainder of the test period.

### Sampling

For each sampling interval (0, 3, 7, 12, and 21 days' storage), duplicate samples for control and DSM32092 test cheese were removed from incubation and assayed by aseptically removing one sample, diluting with peptone water, homogenizing with a Stomacher, and then plating serial dilutions on Listeria selective PALCAM Agar. Listeria monocytogenes was enumerated (cfu/g) on PALCAM agar (30° C., 7 days). Determination of pH of the cheeses during storage was performed (pH meter, Type 1120, Mettler Toledo, combination pH probe INLAB power PRO-ISM, Mettler Toledo, Ohio, USA).

Table 7 shows population of Listeria monocytogenes (LM) and pH change in the control and DSM32092 test Mozzarella cheese. Growth change is also depicted in FIG. 5.

TABLE 7
Growth of Listeria monocytogenes (LM) and pH in the test and control cheese

| | Control Mozzarella Cheese | | | DSM32092 Test Mozzarella Cheese | | |
|---|---|---|---|---|---|---|
| | | Average LM | | | Average LM | |
| | | log (CFU/g) | | | log (CFU/g) | |
| | LM log | change from | | LM log | change from | |
| Time point | (CFU/g) | 0-time | pH | (CFU/g) | 0-time | pH |
| Day 0 | 2.00 | | 5.07 | 1.99 | | 5.04 |
| | 2.00 | | | 2.01 | | |
| Average ± SD | 2.0 ± 0.0 | | | 2.0 ± 0.01 | | |
| Day 3 | 2.47 | | 5.07 | 2.00 | | 5.01 |
| | 2.60 | | | 2.00 | | |
| Average ± SD | 2.54 ± 0.09 | +0.54 | | 2.0 ± 0.00 | +0.00 | |
| Day 7 | 3.17 | | 5.04 | 2.78 | | 5.00 |
| | 3.20 | | | 2.60 | | |
| Average ± SD | 3.19 ± 0.02 | +1.19 | | 2.7 ± 0.12 | +0.70 | |
| Day 12 | 3.34 | | 5.01 | 2.78 | | 4.94 |
| | 3.30 | | | 2.78 | | |
| Average ± SD | 3.32 ± 0.03 | +1.32 | | 2.78 ± 0.00 | +0.77 | |
| Day 21 | 3.30 | | 4.99 | 2.60 | | 4.91 |
| | 3.41 | | | 2.69 | | |
| Average ± SD | 3.36 ± 0.08 | +1.36 | | 2.65 ± 0.07 | +0.65 | |

This example demonstrates that growth inhibition of Listeria monocytogenes can be achieved in Mozzarella cheese using Lactobacillus rhamnosus DSM32092.

1. A method for inhibiting growth of Listeria in cheese, comprising fermenting milk or a milk substrate with an anti-Listerial Lactobacillus rhamnosus strain selected from Lactobacillus rhamnosus strain DSM 32092 deposited at Leibniz Institute DSMZ—German Collection of Microorganisms and Cell Cultures under accession number DSM 32092 and mutants thereof that exhibit at least 75% of the anti-Listerial activity of Lactobacillus rhamnosus strain DSM 32092.

2. The method of claim 1, wherein the Listeria is of species Listeria monocytogenes.

3. The method of claim 1, wherein the cheese is selected from fresh cheese, soft cheese, and semisoft cheese.

4. The method of claim 1, wherein the milk or the milk substrate is inoculated with L. rhamnosus strain DSM 32092 at a concentration of at least 1×10⁶ colony forming units CFU)/ml.

5. The method of claim 1, wherein the milk or the milk substrate is inoculated with a starter culture and the L. rhamnosus strain DSM 32092.

6. The method claim 5, wherein the starter culture is a starter culture comprising one or more of Lactobacillus spp., Streptococcus spp., Leuconostoc spp., and Lactococcus spp.

7. The method of claim 5, wherein the starter culture does not comprise bacteria of species Lactobacillus fermentum.

8-10. (canceled)

11. The method of claim 1, wherein the cheese is selected from cottage cheese, white brined cheese, rindless soft cheese, white mold soft cheese, smear-ripened soft cheese, blue-veined soft cheese and pasta filata cheese.

12. (canceled)

13. A cheese comprising bacteria of an anti-Listerial Lactobacillus rhamnosus strain selected from Lactobacillus rhamnosus strain DSM 32092 and mutants thereof that exhibit at least 75% of the anti-Listerial activity of Lactobacillus rhamnosus strain DSM 32092, wherein the cheese is selected from cottage cheese, white brined cheese, rindless soft cheese, white mold soft cheese, smear-ripened soft cheese, blue-veined soft cheese and pasta filata cheese.

14. The cheese of claim 13, wherein the bacteria of Lactobacillus rhamnosus strain DSM 32092 are present at a concentration of 1×10⁸ CFU/g.

15. The cheese of claim 13, wherein the cheese does not comprise bacteria of species Lactobacillus fermentum.

16. The method of claim 1, wherein the anti-Listerial Lactobacillus rhamnosus strain comprises Lactobacillus rhamnosus strain DSM 32092.

17. The method of claim 1, wherein the milk or the milk substrate is inoculated with L. rhamnosus strain DSM 32092 at a concentration of at least 1×10⁷ CFU/ml.

18. The method of claim 1, wherein the milk or the milk substrate is inoculated with L. rhamnosus strain DSM 32092 at a concentration of at least 1×10⁸ CFU/ml.

19. The method of claim 5, wherein the starter culture comprises Lactococcus lactis subsp. lactis and Streptococcus thermophilus.

20. The method of claim 5, wherein the starter culture consists of Lactococcus lactis subsp. lactis and Streptococcus thermophilus.

21. The cheese of claim 13, wherein the anti-Listerial Lactobacillus rhamnosus strain comprises Lactobacillus rhamnosus strain DSM 32092.

22. The cheese of claim 14, wherein the cheese is cottage cheese or pasta filata cheese.

23. A cheese made by the method of claim 1, wherein the cheese is selected from cottage cheese, white brined cheese, rindless soft cheese, white mold soft cheese, smear-ripened soft cheese, blue-veined soft cheese and pasta filata cheese, and wherein the cheese does not comprise bacteria of species Lactobacillus fermentum.
